(19) United States
(12) Patent Application Publication
(10) Pub. No.: US 2025/0101470 A1
(43) Pub. Date: Mar. 27, 2025

(54) METHOD FOR IMPROVING THE YIELD OF ERINACINE A BY LIQUID FERMENTATION OF HERICIUM ERINACEUS

(71) Applicant: Zhejiang University of Technology, Hangzhou (CN)

(72) Inventors: Peilong SUN, Hangzhou (CN); Kai YANG, Hangzhou (CN); Jian WANG, Hangzhou (CN); Bangwei YE, Hangzhou (CN); Yongjian CHEN, Hangzhou (CN)

(73) Assignee: Zhejiang University of Technology, Hangzhou (CN)

(21) Appl. No.: 18/895,361

(22) Filed: Sep. 24, 2024

(30) Foreign Application Priority Data
Sep. 26, 2023 (CN) 202311247138.7

(51) Int. Cl. C12P 5/00

(52) U.S. Cl. CPC C12P 5/007; C12P 5/002

(57) ABSTRACT

A method for increasing the production of Erinacine A in liquid fermentation of Hericium erinaceus is provided. Through screening of exogenous factors, methyl violet and/or rotenone are selected as the exogenous factors, which ultimately greatly increases the Erinacine A content in the liquid fermentation of Hericium erinaceus, with important production and application value. The liquid fermentation method has high efficiency and can significantly increase the content of Erinacine A in the liquid fermentation process, breaking through the synthetic barrier and having the characteristics of large increase amplitude and fast effect. Compared with other current research on liquid fermentation of Hericium erinaceus to produce Erinacine A, the method for increasing the liquid fermentation production of Erinacine A from Hericium erinaceus is at the highest level, with important research significance.

### CROSS REFERENCE TO THE RELATED APPLICATIONS

This application is based upon and claims priority to Chinese Patent Application No. 202311247138.7, filed on Sep. 26, 2023, the entire contents of which are incorporated herein by reference.

### TECHNICAL FIELD

The present invention belongs to the field of microbial fermentation technology, specifically relates to a method to increase the yield of Erinacine A by liquid fermentation of Hericium erinaceus.

### BACKGROUND

Hericium erinaceus, also known as lion's mane mushroom or monkey head mushroom, is one of the commonly seen rare edible and medicinal fungi belonging to the Tooth Fungusfamily and the Hericium genus. Currently, various nutritional and bioactive substances have been isolated from the fruiting bodies, mycelium and fermentation broth of Hericium erinaceus, which have been found to have beneficial effects such as enhancing the human immune system, anti-tumor properties, protecting the stomach, protecting nerve cells, and preventing neurodegenerative diseases. The main active compounds in Hericium erinaceus are a class of diterpenoid compounds called Erinacines, among which Erinacine A has a relatively high content in the Hericium erinaceus. The biological activities of Erinacine A have been extensively studied, and it has been shown to have significant neuroprotective and anti-tumor effects. Therefore, Hericium erinaceus powder rich in Erinacine A has broad application prospects and high economic value as a raw material for pharmaceuticals or functional foods.

Wild Hericium erinaceus resources are limited, artificial cultivation cycle is long, slow growth and susceptible to environmental factors, with the development and utilization of Hericium erinaceus in health food and medicine, wild resources have been far from meeting the needs of industrial yield, liquid fermentation method can obtain a large number of mycelia and fermentation broth in a short period of time, and the process equipment is relatively simple, the quality of the yield process is easy to control, has become an important way to produce edible and medicinal mushrooms, However, Erinacine A is mainly enriched in the mycelium of Hericium erinaceus, so liquid fermentation is an effective method for the yield of Erinacine A.

Due to the low yield of Erinacine A in Hericium erinaceus and the low extraction efficiency, it has not been well utilized in pharmaceutical development, greatly limiting the research and practical application of Erinacine A. Currently, there is not much research on increasing the yield of Erinacine A, and the main approach is to optimize the liquid fermentation culture medium to achieve higher yields. In the past decade or so, induction regulation technology has become a new research hotspot in liquid fermentation. Induction regulation is a fermentation strategy that uses low-dose inducers to promote the rapid biosynthesis of target metabolites. It has the advantages of high economic benefits, high specificity, and simple process.

As reported in the article “Erinacine A biosynthesis in submerged cultivation of Hericium erinaceus: Quantification and improved cultivation”, published in Engineering in Life Science in 2010 by KRZYCZKOWSKI et al., they used submerged cultivation of Hericium erinaceus (BCRC35669) and optimized the culture medium using a central composite rotatable design (CCRD) method. After optimization, the Erinacine A content in the mycelium reached 14.44 mg/g of dry cell weight, with a yield of 192 mg/L. While optimizing the culture medium can effectively increase the yield of Erinacine A, the improvement is limited. Additionally, since different strains have different characteristics, the optimized culture medium formulation may not have broad adaptability.

As reported in the article “Improvement of erinacine A productivity in Hericium erinaceus mycelia and its neuroprotective bioactivity against the glutamate-insulted apoptosis”, published in LWT-Food Science and Technology in 2015 by Chi-Huang Chang et al., they used submerged cultivation of Hericium erinaceus (BCRC35669) and improved upon the liquid fermentation medium developed by KRZYCZKOWSKI by adding trace amounts of metal ions such as ferrous, copper, zinc, manganese, and nickel. The addition of these metal ions can increase the biomass and the yield of Erinacine A in the liquid fermentation of Hericium erinaceus. The Erinacine A content reached 13.39 mg/g of dry cell weight, with a yield of 16.8 g/L. This cultivation method increased the mycelium biomass, thereby achieving the goal of improving the overall yield. However, the Erinacine A content did not show a significant increase compared to the previous study.

Such as Po-Yu Cheng et al. 2021 in the Fermentation journal published article “Enhanced erinacine A yield by Hericium Erinaceus using solid-state cultivation”, respectively, using solid fermentation and liquid fermentation to culture Hericium erinaceus (MU30296), The content of Erinacine Ain the mycelium obtained in solid-state fermentation culture could reach 165.36 mg/g dry weight, and the yield was 8.3 g/kg medium. The content of Erinacine A in the mycelium of Hericium erinaceus obtained by liquid fermentation was 12.85 mg/g and the yield was 56.80 mg/L. The paper showed that solid fermentation was more conducive to the cultivation of Erinacine A, but the liquid fermentation medium did not contain an appropriate amount of nitrogen source, which was not suitable for the growth and development of Hericium erinaceus mycelium, so the yield of Erinacine A was low. On the other hand, solid-state fermentation is not suitable for factory yield due to its long yield cycle and high raw material costs.

Currently, simply through the optimization of the optimization of the liquid fermentation medium of Hericium erinaceus to increase the yield of Erinacine A technology has been relatively limited. The method of using inducers to promote the synthesis of Erinacine A during fermentation is of great scientific significance to break through the technical barriers of liquid fermentation.

### SUMMARY

The present invention aims to overcome the deficiencies in the prior art, provides a method to increase the yield of Erinacine A by liquid fermentation of Hericium erinaceus, thereby achieving low cost, significantly increasing the content of Erinacine A in the yield process of Hericium erinaceus, efficient and stable, simple process.

To achieve the purpose of the above invention, the present invention is achieved by the following technical solutions:

A method for improving the yield of Erinacine A by liquid fermentation of Hericium erinaceus comprises the following steps:
(S.1) The Hericium erinaceus strain was inoculated on the potato dextrose agar (PDA) slope medium for activation to obtain the slope strains;
(S.2) the slope strains obtained in step (S.1) were inoculated into liquid seed medium for culture to obtain Hericium erinaceus liquid seeds;
(S.3) the liquid seeds obtained in step (S.2) were inoculated into the liquid fermentation medium for liquid fermentation culture, and an inducer was added in the liquid fermentation process to induce fermentation until the end of the reaction, and the mycelium of Hericium erinaceus was obtained after filtration and drying;
(S.4) The Hericium erinaceus mycelium obtained in step (S.3) is crushed and added to methanol, heated and ultrasonicated, the supernatant is centrifuged, and an alcohol extract containing Erinacine A is obtained after filtration.

This invention uses Hericium erinaceus for liquid fermentation, and adds a suitable amount of inducer during the liquid fermentation process. After the induced fermentation is completed, the Hericium erinaceus mycelium are collected, dried, and pulverized. Methanol is then added to the dried and powdered Hericium erinaceus mycelium, and ultrasound-assisted extraction is performed. The alcohol extract is then analyzed by high-performance liquid chromatography. The results show that: After 10 days of fermentation with the addition of 5 mg/L methyl viologen solution, the Erinacine A content in the Hericium erinaceus mycelium increased from 0.19±0.05 mg/g dry mycelium to 21.94±2.40 mg/g dry mycelium, an increase of about 115.47 times. The Erinacine A yield increased from 4.48±0.96 mg/L to 414.84±30.03 mg/L, an increase of about 92.60 times. After 8 days of fermentation with the addition of 5 mg/L rotenone ethanol solution, the Erinacine A content in the Hericium erinaceus mycelium increased from 0.19±0.05 mg/g dry mycelium to 0.67±0.04 mg/g dry mycelium, an increase of about 2.52 times. The Erinacine A yield increased from 4.48±0.96 mg/L to 13.72±0.24 mg/L, an increase of about 2.06 times.

Methyl viologen, also known as paraquat, is a pesticide ingredient. The toxicity of methyl viologen (MV) to living organisms is mainly due to the oxidative stress response caused by the reactive oxygen species (ROS) generated by cellular reactions. In non-photosynthetic organisms, MV primarily generates ROS in the mitochondria, where it typically acts as an electron acceptor for oxidoreductase enzymes, accepting electrons and participating in the electron transport process. When plants face external stressors, they mount their own defense response, activating a series of antioxidant reactions and producing secondary metabolites to scavenge ROS, with Erinacine A potentially being a major product synthesized in this process. Studies have shown that rotenone alters the abundance of amino acid metabolism pathways and types in fungi, which may be beneficial for the biosynthesis of Erinacine A, but the specific mechanism of rotenone's action on fungi is still unclear. The liquid fermentation method of this invention has high efficiency and can significantly increase the content of Erinacine A in the liquid fermentation yield process of Hericium erinaceus.

Preferably, the liquid seed culture medium composition in step (S.2) is:

Glucose 25-35 g, soybean meal powder 15-30 g, potassium dihydrogen phosphate 0.5-2 g, magnesium sulfate 0-1 g, the rest is water.

Further preferably, the PDA slant culture medium composition in step (S.1) is:

Potato extract, glucose 15-25 g, agar powder 10-15 g, made up to 1 L with water.

Even more preferably, the method for extracting the potato extract is:

200 g peeled potatoes are cut into thin slices, boiled in water for 30 minutes, filtered through a gauze, and made up to 1 L to obtain the potato extract.

Even more preferably, the activation temperature of the PDA slant culture medium in step (S.1) is 25-28° C.

Preferably, the ratio of the inoculation volume of the Hericium erinaceus liquid seed to the volume of the liquid fermentation culture medium in step (S.3) is 8-12.5%.

When step (S.3) in the Hericium erinaceus liquid seed inoculation volume and liquid fermentation medium volume ratio (i.e. Hericium erinaceus liquid seed inoculum) is less than 8% (v/v), easy to cause mycelium in the fermentation broth growth rate slowed down, fermentation cycle prolonged, insufficient growth, and further affect the fermentation efficiency and reduce yield. When the ratio of the inoculation volume of liquid seeds to the volume of liquid fermentation medium in step (S.3) is higher than 12.5% (v/v), it is easy to lead to excessive mycelium growth, insufficient nutrient supply, fierce oxygen competition, and large accumulation of metabolites, thereby reducing the fermentation efficiency and quality.

Preferably, the inducer in the step (S.3) is methyl viologen, rotenone in any one or a combination of two.

Preferably, the inducer added in the step (S.3) in the liquid fermentation medium concentration of 5-20 mg/L.

When the concentration of the inducer added in the step (S.3) is less than 5 mg/L, may not be able to effectively trigger the induction reaction of mycelium, resulting in the retardation of the synthesis and metabolic process of related enzymes, thereby affecting the growth and metabolism of mycelium. As a result, the induction effect is reduced, which in turn affects the formation of fermentation products. When the concentration of the inducer added in step (S.3) is higher than 20 mg/L, it is easy to inhibit the growth and metabolism of mycelium, and may even have a toxic effect on mycelium, resulting in problems such as mycelium death or growth arrest. In addition, too high inducer concentrations may interfere with the action of other fermentation factors, thus affecting the normal progress of the liquid fermentation process.

Preferably, the steps (S.3) in the liquid fermentation culture 4-7 days to add an inducer.

When step (S.3) in the liquid fermentation culture less than 3 days when the inducer is added, the mycelium has not yet fully grown and multiplied, the sensitivity to the inducer is low, the induction effect is not good. In addition, the premature addition of inducers may interfere with the normal growth and metabolism of mycelium, affecting the efficiency and quality of fermentation. When the inducer is added in step (S.3) when the liquid fermentation culture is carried out for more than 6 days, the mycelium has grown sufficiently and entered the peak metabolic period, and the effect of the inducer is easily inhibited, which then affects the induction effect. In addition, late addition of inducers can easily lead to excessive metabolism of mycelium, which affects the generation of fermentation products and the degradation of quality.

Preferably, the reaction time for induction fermentation in the described step (S.3) is 2-6 days.

When step (S.3) in the induction of fermentation reaction time less than 2 days, mycelium may not have been sufficiently induced and respond to the action of inducers, fermentation reaction is not complete, thereby affecting the generation of fermentation products and quality reduction. When the reaction time of induction fermentation in step (S.3) is more than 6 days, too long induction time can easily lead to excessive metabolites produced by mycelium, and excessive metabolites may interact with each other or produce toxicity, inhibit the growth and metabolism of mycelium, thereby affecting the fermentation efficiency and quality.

Preferably, the liquid fermentation medium components in the steps (S.3) are:

Glucose 25-35 g, yeast powder 15-30 g, potassium dihydrogen phosphate 0.5-2 g, magnesium sulfate 0-1 g, the remaining amount is water.

Preferably, the steps (S.2) as well as the steps (S.3) in the medium of culture conditions are:

Incubate at 25-28° C. and 130-160 rpm for 1-10 days with shaking.

Preferably, the reaction conditions for the ultrasonication of Erinacine Ain the steps (S.4) are:

The ultrasonic power is 200-350 W, the heating reaction temperature is 50-70° C., and the ultrasonic reaction time is 40-90 min.

Therefore, the present invention has the following beneficial effects:
(1) The present invention is screened by exogenous factors, and methyl viologen and/or rotenone are selected as exogenous factors, and finally the content of Erinacine A produced by liquid fermentation of Hericium erinaceus is greatly improved, which has important yield and application value;
(2) the liquid fermentation method of the present invention has high efficiency, can significantly improve the content of Erinacine A in the liquid fermentation yield process of Hericium erinaceus, breaks through the synthetic barrier, and has the characteristics of large improvement range and quick effect;
(3) Compared with other current research work on the yield of Hericium erinaceus by liquid fermentation, the method of improving the yield of Erinacine A by liquid fermentation of Hericium erinaceus in the present invention is at the highest level and has important research significance.

### BRIEF DESCRIPTION OF THE DRAWINGS

FIG. 1 is the molecular structure diagram of Erinacine A.

FIG. 2 is the process flow diagram for increasing the yield of Erinacine A by liquid fermentation of Hericium erinaceus.

FIG. 3 is the retention time diagram of the Erinacine A standard and sample.

FIG. 4 is the standard curve diagram of Erinacine A.

FIG. 5 is the HPLC detection diagram of the blank control group without adding any inducer.

FIG. 6 is the HPLC detection diagram after methyl viologen treatment.

FIG. 7 is the HPLC detection diagram after rotenone treatment.

### DETAILED DESCRIPTION OF THE EMBODIMENTS

The present invention is further described below in conjunction with the drawings of the description and specific examples. A person of ordinary skill in the art would be able to realize the present invention based on these descriptions. In addition, the examples of the present invention referred to in the following description are usually only examples of a part of the present invention, but not all examples. Therefore, based on the examples in the present invention, all other examples obtained by a person skilled in the art without creative labor shall fall within the scope of protection of the present invention.

### Example 1

The strain number of a Hericium erinaceus strain is ACCC50268; source: China Agricultural Culture Collection and Management Center.

A method for improving the liquid fermentation yield of Erinacine A from Hericium erinaceus, including the following steps:

(S.1) A strain of Hericium erinaceus was subcultured for 3 generations to achieve strain activation. The subculture temperature is 25-28° C., and the optimal culture temperature in this example is 28° C., and the time is 14 days, so that the entire inclined plane can be grown to obtain a slope strain. The components of PDA inclined medium are: potato extract, 15-25 g of glucose, 10-15 g of agar powder, and the volume is set to 1 L with water. In this example, the optimal PDA inclined medium components are as follows: potato extract, 20 g of glucose, 15 g of agar powder, and the volume is determined to 1 L with water;

The preparation method of PDA inclined medium is as follows:

Take 200 g of potatoes and peel them, cut them into thin pieces, add distilled water, boil them for 30 min, filter them with gauze, filter out the potato pieces, and obtain potato extract. 20 g of glucose and 15 g of agar powder were added to the potato extract, and the filtrate was supplemented to 1 L with water to obtain PDA medium. The PDA medium was divided into each test tube, sterilized by an autoclave at 121° C. for 30 min, and then cooled into a PDA inclined medium.

(S.2) The slope strains obtained in step (S.1) were inoculated into the primary liquid seed medium for culture. One vial of bevel strains was inoculated into two vials of primary liquid seed medium. The components of liquid seed medium are: glucose 25-35 g, soybean meal powder 15-30 g, potassium dihydrogen phosphate 0.5-2 g, magnesium sulfate 0-1 g, and the balance is water. The culture conditions of liquid seed medium are: 1-10 days at 25-28° C. and 130-160 rpm in the dark. In this example, the optimal liquid seed medium components are as follows: glucose 30 g, soybean meal powder 20 g, potassium dihydrogen phosphate 1 g, magnesium sulfate 0.5 g, and the balance is water. The medium was fixed to 1 L with purified water, and the prepared liquid seed medium was loaded into a 500 mL Erlenmeyer flask with a liquid volume of 200 mL, and cultured at 27° C. and 140 rpm for 7 days in the dark, and the liquid seeds of Hericium erinaceus were obtained.

(S.3) the liquid seeds obtained in step (S.2) are inoculated into the liquid fermentation medium for liquid fermentation culture, the ratio of the liquid seed inoculation volume of Hericium erinaceus to the volume of the liquid fermentation medium is 8-12.5% (i.e., the inoculation amount of Hericium erinaceus liquid seeds is 8-12.5%), and the optimal inoculation amount of Hericium erinaceus liquid seeds in this example is 10%. The components of liquid fermentation medium are: glucose 25-50 g, yeast powder 10-30 g, potassium dihydrogen phosphate 0.5-2 g, magnesium sulfate 0-1 g, and the balance is water. The culture conditions of liquid fermentation medium are: 1-10 days at 25-28° C. and 130-160 rpm in the dark. In this example, the optimal liquid fermentation medium components are as follows: 30 g of glucose, 30 g of yeast powder, 1 g of potassium dihydrogen phosphate, 0.5 g of magnesium sulfate, and the balance is water. The medium was fixed to 1 L with distilled water, and the prepared liquid seed medium was packed into a 500 mL Erlenmeyer flask with a liquid volume of 200 mL, and incubated at 27° C. and 140 rpm for 7 days in the dark.

weigh 50 mg methyl viologen (purity greater than 96%) in a beaker, dissolve it in a small amount of distilled water, transfer it to a 50 mL volumetric flask, and set the volume with distilled water to prepare 1 mg/mL methyl viologen solution; weigh 50 mg rotenone (purity greater than 95%) in a beaker, dissolve it in a small amount of 95% ethanol, transfer it to a 50 mL volumetric flask, set the volume with 95% ethanol, and prepare it into 1 mg/mL rotenone solution;

On the 5th day of inoculating the liquid seeds of Hericium erinaceus obtained in step (S.2) into liquid fermentation medium for liquid fermentation, 1 mL of methyl viologen solution was added to its concentration in liquid fermentation medium to a concentration of 5 mg/L. Induction fermentation was continued, and the reaction ended after 6 days of induction fermentation. After the end of fermentation, the medium was filtered through 80 mesh gauze, washed with water three times, and then dried overnight at 60° C. with hot air to obtain Hericium erinaceus mycelium;

(S.4) The Hericium erinaceus mycelium obtained in step (S.3) was crushed, and 1 g of Hericium erinaceus mycelium dry powder was added to 20 mL of 80% methanol, heated and ultrasonicated. The reaction conditions for ultrasonic extraction of Erinacine A are as follows: ultrasonic power is 200-350 W, heating reaction temperature is 50-70° C., and ultrasonic reaction time is 40-90 min. In this example, the reaction conditions for the optimal ultrasonic extraction of Erinacine A are as follows: ultrasonic extraction at 60° C. and ultrasonic power of 300 W for 60 min. The supernatant was centrifuged to obtain an alcohol extract containing Erinacine A. The alcohol extract was filtered through a 0.22 μm filter membrane and then analyzed in liquid phase.

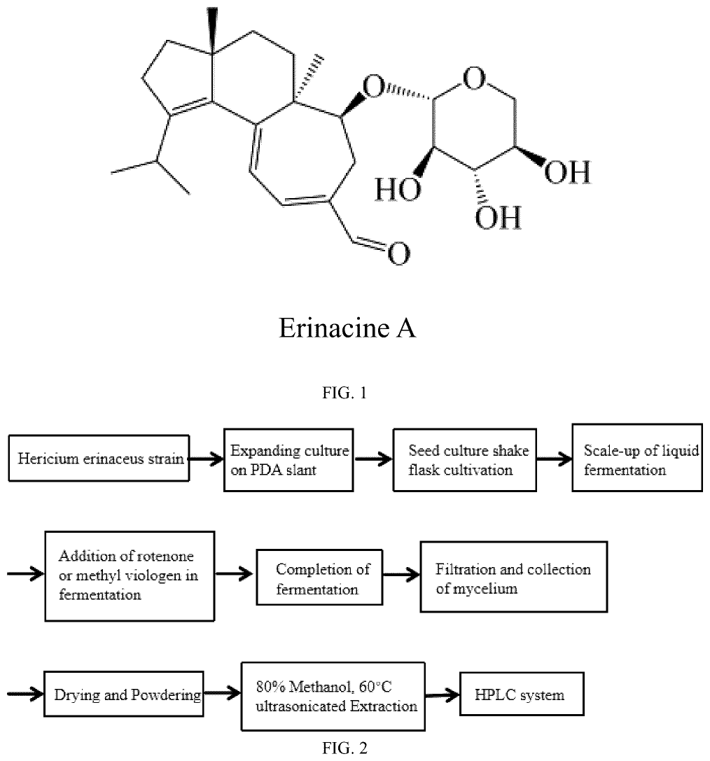

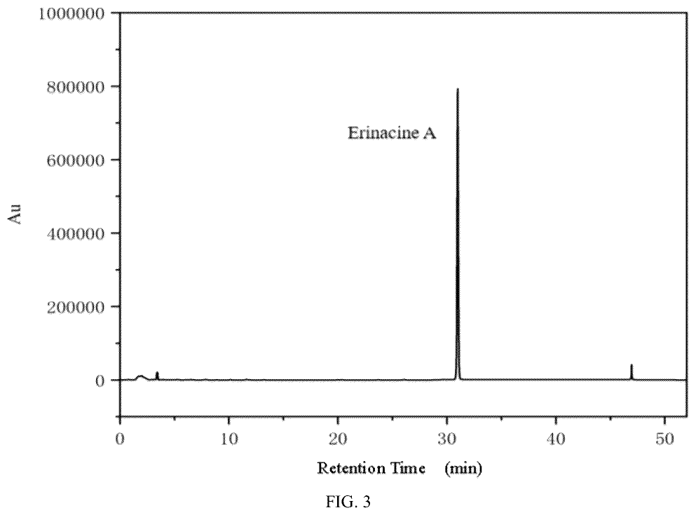

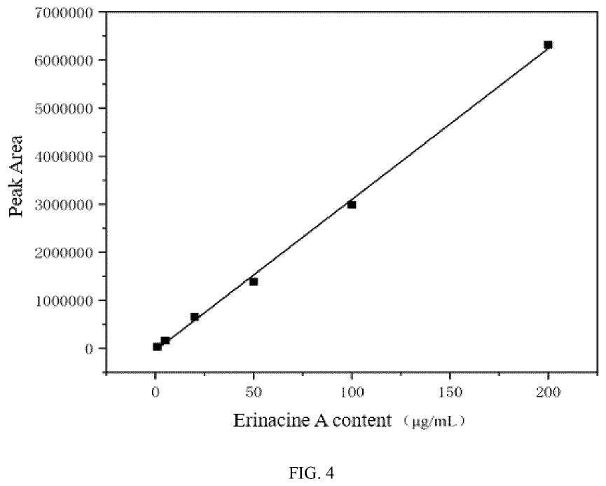

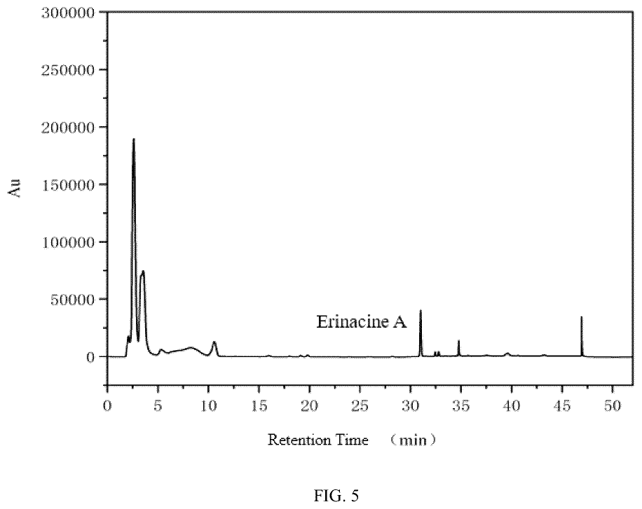

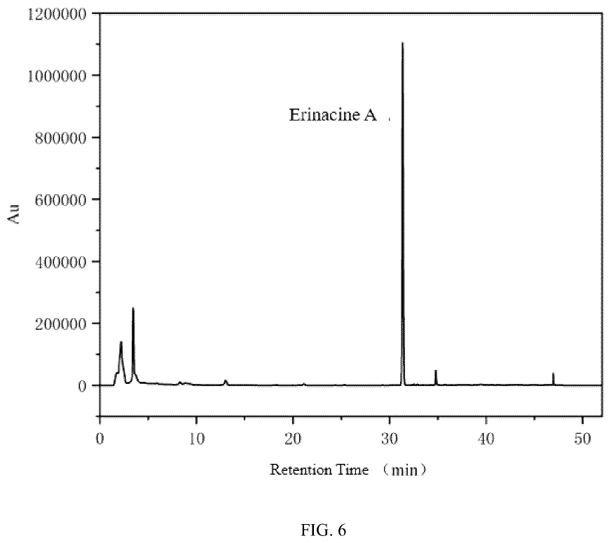

The detection conditions of high-performance liquid chromatography were as follows: SHIMAZU LC2030 HPLC system, using Phenomenex luna C18 column (4.6×250 mm, 5 μm); Mobile phase: composed of water and acetonitrile, 0-23 min, 30% acetonitrile-50% acetonitrile; 23-26 min, 50% acetonitrile, 26-32 min, 50% acetonitrile-100% acetonitrile, 32-44 min, 100% acetonitrile, 44-60 min, 30% acetonitrile; Column temperature 30° C.; Flow rate: 1.0 mL/s; Injection volume 20 μL; The detection wavelength is 340 nm. The peak time of Erinacine A was about 31.2 min. The molecular structure of Erinacine A is shown in FIG. 1. The process flow chart for improving the yield of Erinacine A by liquid fermentation is shown in FIG. 2. The standards for Erinacine A, as well as the sample retention times, are shown in FIG. 3. The standard curve for Erinacine A is shown in FIG. 4. The high-performance liquid chromatography (HPLC) detection image after methyl viologen refining is shown in FIG. 6.

### Example 2

The example is different from example 1 in that:

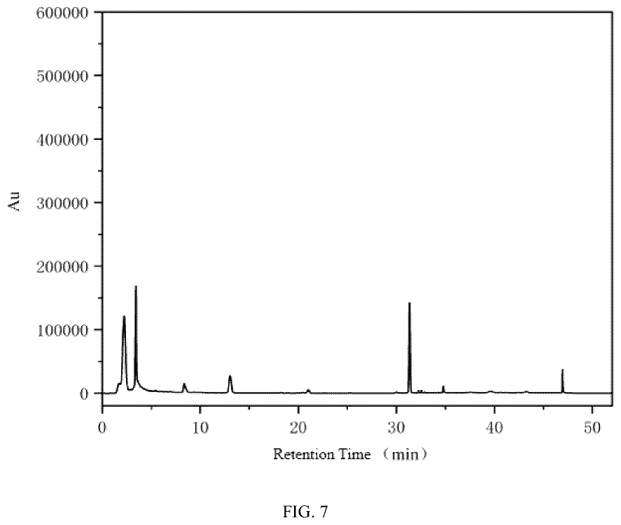

In this example, a method for improving the yield of Hericium erinaceus by liquid fermentation is provided, wherein 1 mL of rotenone solution is added to replace 1 mL of methyl viologen solution on the 5th day of liquid fermentation culture in step (S.3). Everything else is the same as in Example 1. The HPLC detection image after rotenone treatment is shown in FIG. 7.

### Example 3

The example is different from example 1 in that:

In this example, a method for improving the yield of Hericium erinaceus by liquid fermentation is provided, wherein 1 mL of methyl viologen solution is added on the 4th day of liquid fermentation culture in step (S.3). Everything else is the same as in Example 1.

### Example 4

The example is different from example 1 in that:

In this example, a method for improving the yield of Hericium erinaceus from liquid fermentation is provided, wherein 1 mL of methyl viologen solution is added on the 6th day of liquid fermentation culture in step (S.3). Everything else is the same as in Example 1.

### Example 5

The example is different from example 1 in that:

In this example, a method for improving the yield of Hericium erinaceus by liquid fermentation is provided, wherein 1 mL of methyl viologen solution is added on the 7th day of liquid fermentation culture in step (S.3). Everything else is the same as in Example 1.

### Example 6

The example is different from example 1 in that:

In this example, a method for increasing the yield of Hericium erinaceus from liquid fermentation is provided, wherein induction fermentation is continued in step (S.3), and the reaction is completed after 2 days of induction fermentation. Everything else is the same as in Example 1.

### Example 7

The example is different from example 1 in that:

In this example, a method for increasing the yield of Hericium erinaceus from liquid fermentation is provided, wherein the induction fermentation is continued in step (S.3), and the reaction is terminated after 3 days of induction fermentation. Everything else is the same as in Example 1.

### Example 8

The example is different from example 1 in that:

In this example, a method for increasing the yield of Erinacine A by liquid fermentation of Hericium erinaceus is provided, wherein the induction fermentation is continued in step (S.3), and the reaction is terminated after 4 days of induction fermentation. Everything else is the same as in Example 1.

### Example 9

The example is different from example 1 in that:

In this example, a method for increasing the yield of Hericium erinaceus from liquid fermentation is provided, wherein induction fermentation is continued in step (S.3), and the reaction ends after 5 days of induction fermentation. Everything else is the same as in Example 1.

### Example 10

The example is different from example 1 in that:

In this example, a method for increasing the yield of Hericium erinaceus by liquid fermentation is provided, wherein 2 mL of methyl viologen solution is added to step (S.3) so that its concentration in liquid fermentation medium is 10 mg/L. Everything else is the same as in Example 1.

### Example 11

The example is different from example 1 in that:

In this example, a method for increasing the yield of Hericium erinaceus from liquid fermentation is provided, wherein 4 mL of methyl viologen solution is added in step (S.3) to make its concentration in liquid fermentation medium 20 mg/L. Everything else is the same as in Example 1.

### Proportion 1

The difference between the ratio of this pair and the example 1 is that:

In this pair ratio, a method is provided to increase the yield of Erinacine A by liquid fermentation, in which no exogenous factors, i.e., no inducers, were added on the 5th day of liquid fermentation culture in step (S.3) as a blank control group. Everything else is the same as in Example 1. The HPLC detection plot of the blank control group without any inducer is shown in FIG. 5.

### Proportion 2

The difference between the ratio of this pair and the example 1 is that:

In this pair, a method is provided to increase the yield of Erinacine A by liquid fermentation of Hericium erinaceus, wherein a methyl viologen solution at a concentration of 5 mg/L in liquid fermentation medium is added on the third day of liquid fermentation culture in step (S.3). Everything else is the same as in Example 1.

### Proportion 3

The difference between the ratio of this pair and the example 1 is that:

In this pair ratio, a method is provided to increase the yield of Erinacine A by liquid fermentation, in which a methyl viologen solution with a concentration of 5 mg/L in liquid fermentation medium is added on the 8th day of liquid fermentation culture in step (S.3).

Everything else is the same as in Example 1.

### Proportion 4

The difference between the ratio of this pair and the example 1 is that:

In this pair, a method is provided to increase the yield of Hericium erinaceus from liquid fermentation of Hericium erinaceus, in which induction fermentation is continued in step (S.3), and the reaction is completed after 1 day of induction fermentation. Everything else is the same as in Example 1.

### Proportion 5

The difference between the ratio of this pair and the example 1 is that:

In this pair ratio, a method is provided to increase the yield of Hericium erinaceus from liquid fermentation, in which the induction fermentation is continued in step (S.3), and the reaction is completed after 8 days of induction fermentation. Everything else is the same as in Example 1.

### Proportion 6

The difference between the ratio of this pair and the example 1 is that:

In this pair ratio, a method is provided to increase the yield of Hericium erinaceus from liquid fermentation of Hericium erinaceus, in which 0.2 mL of methyl viologen solution is added in step (S.3) to make its concentration in liquid fermentation medium 1 mg/L. Everything else is the same as in Example 1.

### Proportion 7

The difference between the ratio of this pair and the example 1 is that:

In this pair ratio, a method is provided to increase the yield of Erinacine A by liquid fermentation of Hericium erinaceus, wherein 10 mL of methyl viologen solution is added in step (S.3) to a concentration of 50 mg/L in liquid fermentation medium. Everything else is the same as in Example 1.

### Proportion 8

The difference between the ratio of this pair and the example 1 is that:

In this pair ratio, a method is provided to increase the yield of Erinacine A by liquid fermentation, in which 1 mL of oleic acid (purity greater than 95%) is added to replace 1 mL of methyl viologen solution on the 5th day of liquid fermentation culture in step (S.3). Everything else is the same as in Example 1.

### Proportion 9

The difference between the ratio of this pair and the example 1 is that:

In this pair ratio, a method is provided to increase the yield of Hericium erinaceus from liquid fermentation of Hericium erinaceus, in which 1 mL of sweet orange oil (purity greater than 95%) is added to replace 1 mL of methyl viologen solution on the 5th day of liquid fermentation culture in step (S.3). Everything else is the same as in Example 1.

### Performance Testing

The alcohol extract containing Erinacine A is obtained by processing according to the method in example 1-2 and the proportion 1 and the proportion 8-9 respectively. The alcohol extract was filtered through a 0.22 μm filter membrane and then analyzed in liquid phase. The effect of Hericium erinaceus liquid fermentation on the production of Erinacine A is shown in Table 1 below.

TABLE 1

| | Mycelium | Erinacine A content | Erinacine A |
|---|---|---|---|
| Constituencies | yield (g/L) | (mg/g dried mycelium) | yield (mg/L) |
| Example 1 | 18.96 ± 0.69 | 94.94 ± 2.40 | 414.84 ± 30.03 |
| Example 2 | 8.50 ± 1.69 | 0.67 ± 0.04 | 13.72 ± 0.24 |
| Proportion 1 | 23.79 ± 1.70 | 0.19 ± 0.05 | 4.48 ± 0.96 |
| Proportion 8 | 23.72 ± 1.04 | 0.20 ± 0.10 | 4.70 ± 2.16 |
| Proportion 9 | 17.51 ± 1.12 | 0.06 ± 0.01 | 1.18 ± 0.23 |

From Table 1, FIGS. 3-7 data analysis can be seen: in the 4th day of liquid fermentation culture do not add any exogenous factors, that is, do not add any inducers, as a blank control group (to the proportion 1). After 6 days of liquid fermentation, the mycelium of Hericium erinaceus were filtered, dried, and ground and then extracted with 80% methanol ultrasonic heating. The content of Erinacine A in the blank control group (ratio 1) was 0.19±0.05 mg/g, and the yield of Erinacine A was 4.48±0.96 mg/L.

On the 5th day of liquid fermentation added in the liquid fermentation medium with a concentration of 5 mg/L methyl viologen essence solution, induced fermentation 6 days later, Hericium erinaceus mycelium filtered, dried, ground with 80% methanol ultrasonic heating assisted extraction, methyl viologen refining treatment group (example 1) of the Erinacine A content increased from 0.19 mg/g to 21.94±2.40 mg/g, The yield of Erinacine A increased from 4.48 mg/L to 414.84±30.03 mg/L, and the addition of methyl viologen significantly increased the yield of Erinacine A.

On the 5th day of liquid fermentation added in the liquid fermentation medium with a concentration of 5 mg/L rotenone solution for induction treatment, induced fermentation 6 days later, Hericium erinaceus mycelium filtered, dried, ground with 80% methanol ultrasonic heating assisted extraction, For the rotenone treatment group (example 2), the content of Erinacine A increased from 0.19 mg/g to 0.67 mg/g, The yield of Erinacine A increased from 4.48 mg/L to 13.72 mg/L, and the addition of rotenone treatment also had a positive effect on the increase of Erinacine A yield, but the improvement effect was not as significant as that of methyl viologen. The effects of different exogenous factors were compared, and it was found that the addition of 1 mL oleic acid (Proportion 8) or 1 mL sweet orange oil (Proportion 9) on the 5th day of liquid fermentation for induction treatment did not show obvious stimulating effects, and even the sweet orange oil inhibited the synthesis of Erinacine A.

Respectively, in accordance with the example 1, example 3-5 and the proportion of 2-3 in the method of processing to obtain an alcohol extract containing Erinacine A. The alcohol extract was filtered through a 0.22 μm filter membrane and then analyzed in liquid phase. The effect of Hericium erinaceus liquid fermentation on the production of Erinacine A is shown in Table 2 below.

TABLE 2

| | Mycelium | Erinacine A content | Erinacine A |
|---|---|---|---|
| Constituencies | yield (g/L) | (mg/g dried mycelium) | yield (mg/L) |
| Example 3 | 2.00 ± 0.84 | 1.52 ± 0.30 | 22.18 ± 3.84 |
| Example 1 | 18.96 ± 0.69 | 94.94 ± 2.40 | 414.84 ± 30.03 |
| Example 4 | 23.05 ± 1.08 | 2.98 ± 1.16 | 69.29 ± 30.01 |
| Example 5 | 23.22 ± 1.93 | 2.68 ± 0.66 | 61.60 ± 10.06 |
| Proportion 2 | 8.64 ± 1.54 | 0.05 ± 0.01 | 0.41 ± 0.06 |
| Proportion 3 | 23.28 ± 0.57 | 0.57 ± 0.12 | 13.29 ± 2.55 |

From the data analysis in Table 2 it can be seen that: by adding methyl viologen essence solution at different times, it is found that methyl viologen essence has an inhibitory effect on the liquid fermentation growth of Hericium erinaceus. Among them, the addition of methyl viologen on the 5th day of liquid fermentation had the best effect, probably because the growth of mycelium was close to saturation and the synthesis of metabolites was more active during this period. However, after 7 days of liquid fermentation, the mycelium growth reached the maturity stage and the decline stage, during which the metabolic activity of the mycelium decreased, and the effect of inducing the synthesis of Erinacine A was not good.

In accordance with the example 1, example 6-9 and the proportion of 4-5 in the method of processing to obtain an alcohol extract containing Erinacine A. The alcohol extract was filtered through a 0.22 μm filter membrane and then analyzed in liquid phase. The effect of Hericium erinaceus liquid fermentation on the production of Erinacine A is shown in Table 3 below.

TABLE 3

| | Mycelium | Erinacine A content | Erinacine A |
|---|---|---|---|
| Constituencies | yield (g/L) | (mg/g dried mycelium) | yield (mg/L) |
| Example 6 | 17.94 ± 1.68 | 0.68 ± 0.27 | 12.53 ± 3.82 |
| Example 7 | 17.68 ± 0.50 | 2.29 ± 0.52 | 40.30 ± 7.52 |
| Example 8 | 8/20 ± 2.12 | 5.49 ± 1.60 | 103.99 ± 18.67 |
| Example 9 | 5.45 ± 1.39 | 16.58 ± 0.43 | 276.77 ± 4.72 |
| Example 1 | 18.96 ± 0.69 | 94.94 ± 2.40 | 418.84 ± 30.03 |
| Proportion 4 | 20.16 ± 0.85 | 0.07 ± 0.01 | 1,36 ± 0.03 |
| Proportion 5 | 19.79 ± 0.76 | 8.97 ± 2.33 | 179.50 ± 62.29 |

From the data analysis in Table 3, it can be seen that the induction time after the addition of methyl viologen, the results show that after the addition of methyl viologen, the yield of Erinacine A increases with time, and the synthesis rate of Erinacine A increases the fastest when the duration of induction fermentation is 4-6 days, reaching the highest at 6 days of induction fermentation. By prolonging the induction fermentation time, the yield of Erinacine A decreased significantly after 8 days of induction fermentation, but the yield of Erinacine A was still at a high level at this time.

In accordance with the example 1, example 10-11 and the proportion of 6-7 in the method of processing to obtain an alcohol extract containing Erinacine A. The alcohol extract was filtered through a 0.22 μm filter membrane and then analyzed in liquid phase. The effect of Hericium erinaceus liquid fermentation on the production of Erinacine A is shown in Table 4 below.

TABLE 4

| | Mycelium | Erinacine A content | Erinacine A |
|---|---|---|---|
| Constituencies | yield (g/L) | (mg/g dried mycelium) | yield (mg/L) |
| Example 1 | 18.96 ± 0.69 | 94.94 ± 2.40 | 418.84 ± 30.03 |
| Example 10 | 7.40 ± 0.36 | 20.61 ± 3.21 | 400.44 ± 69.30 |
| Example 11 | 19.61 ± 0.48 | 1.50 ± 0.38 | 29.47 ± 7.44 |
| Proportion 6 | 9.49 ± 2.94 | 0.63 ± 0.09 | 13.93 ± 4.56 |
| Proportion 7 | 17.71 ± 1.29 | 0.21 ± 0.04 | 3.71 ± 1.06 |

From the data analysis in Table 4 it can be seen that: under the conditions of optimal addition time and induction duration, the optimal dose of methyl viologen to induce liquid fermentation of Hericium erinaceus is optimized. The results showed that the effect of inducing the synthesis of Erinacine A was the best when the concentration of methyl viologen in liquid fermentation medium was 5-10 mg/L, and the yield of Erinacine A was between 400.44 mg/L and 414.84 mg/L. When the concentration of methyl viologen in the liquid fermentation medium was low, the stress reaction was not intense enough. However, when the concentration of methyl viologen in the liquid fermentation medium is too high, most of the Hericium erinaceus will die or be inactivated, so that the synthesis of Erinacine A cannot be carried out.

The above is only a detailed description of the preferred examples and principles of the present invention, for those skilled in the art, according to the ideas provided by the present invention, there will be changes in the specific examples, and these changes should also be regarded as the scope of protection of the present invention.

What is claimed is:

1. A method for improving a production of Erinacine A by a liquid fermentation of Hericium erinaceus, comprising the following steps:
(S.1) inoculating a Hericium erinaceus strain on a potato dextrose agar (PDA) slope medium for an activation to obtain a slope strain;
(S.2) inoculating the slope strain obtained in the step (S.1) into a liquid seed medium for a culture to obtain Hericium erinaceus liquid seeds;
(S.3) inoculating the Hericium erinaceus liquid seeds obtained in the step (S.2) into a liquid fermentation medium for a liquid fermentation culture, adding an inducer in a liquid fermentation process to induce a fermentation until an end of a reaction, and obtaining a mycelium of the Hericium erinaceus after filtration and drying; and
(S.4) crushing and adding the mycelium of the Hericium erinaceus obtained in the step (S.3) to methanol, performing a heating and an ultrasonic extraction, centrifuging to obtain a supernatant, and filtering the supernatant to obtain an alcohol extract containing the Erinacine A.

2. The method for improving the production of the Erinacine A by the liquid fermentation of the Hericium erinaceus according to claim 1, wherein a liquid seed medium component in the step (S.2) is:
25-35 g of glucose, 15-30 g of soybean meal powder, 0.5-2 g of potassium dihydrogen phosphate, 0-1 g of magnesium sulfate, a remaining amount is water.

3. The method for improving the production of the Erinacine A by the liquid fermentation of the Hericium erinaceus according to claim 1, wherein a ratio of an inoculation volume of the Hericium erinaceus liquid seeds in the step (S.3) to a volume of the liquid fermentation medium is 8-12.5%.

4. The method for improving the production of the Erinacine A by the liquid fermentation of the Hericium erinaceus according to claim 1, wherein the inducer in the step (S.3) is at least one of methyl viologen and rotenone.

5. The method for improving the production of the Erinacine A by the liquid fermentation of the Hericium erinaceus according to claim 4, wherein a concentration of the inducer added in the step (S.3) in the liquid fermentation medium is 5-20 mg/L.

6. The method for improving the production of the Erinacine A by the liquid fermentation of the Hericium erinaceus according to claim 1, wherein the inducer is added on 4-7th day of the liquid fermentation culture in the step (S.3).

7. The method for improving the production of the Erinacine A by the liquid fermentation of the Hericium erinaceus according to claim 1, wherein a reaction time of the fermentation in the step (S.3) is 2-6 days.

8. The method for improving the production of the Erinacine A by the liquid fermentation of the Hericium erinaceus according to claim 1, wherein a liquid fermentation medium component in the step (S.3) is:
25-35 g of glucose, 15-30 g of yeast powder, 0.5-2 g of potassium dihydrogen phosphate, 0-1 g of magnesium sulfate, a remaining amount is water.

9. The method for improving the production of the Erinacine A by the liquid fermentation of the Hericium erinaceus according to claim 1, wherein culture conditions of the liquid seed medium in the step (S.2) and the liquid fermentation medium in the step (S.3) are:
incubate at 25-28° C. and 130-160 rpm for 1-10 days with shaking.

10. The method for improving the production of the Erinacine A by the liquid fermentation of the Hericium erinaceus according to claim 1, wherein reaction conditions for the ultrasonic extraction of the Erinacine A in the step (S.4) are as follows:
an ultrasonic power is 200-350 W, a heating reaction temperature is 50-70° C., and an ultrasonic reaction time is 40-90 min.

11. The method for improving the production of the Erinacine A by the liquid fermentation of the Hericium erinaceus according to claim 3, wherein the inducer in the step (S.3) is at least one of methyl viologen and rotenone.

12. The method for improving the production of the Erinacine A by the liquid fermentation of the Hericium erinaceus according to claim 11, wherein a concentration of the inducer added in the step (S.3) in the liquid fermentation medium is 5-20 mg/L.

13. The method for improving the production of the Erinacine A by the liquid fermentation of the Hericium erinaceus according to claim 2, wherein culture conditions of the liquid seed medium in the step (S.2) and the liquid fermentation medium in the step (S.3) are:
incubate at 25-28° C. and 130-160 rpm for 1-10 days with shaking.

14. The method for improving the production of the Erinacine A by the liquid fermentation of the Hericium erinaceus according to claim 8, wherein culture conditions of the liquid seed medium in the step (S.2) and the liquid fermentation medium in the step (S.3) are:
incubate at 25-28° C. and 130-160 rpm for 1-10 days with shaking.
